(19) United States
(12) Patent Application Publication
(10) Pub. No.: US 2022/0296702 A1
(43) Pub. Date: Sep. 22, 2022

(54) PHARMACEUTICAL COMPOSITIONS COMPRISING ONCOLYTIC HERPES SIMPLEX VIRUS FOR SYSTEMIC ADMINISTRATION

(71) Applicant: IMMVIRA CO., LIMITED, Shenzhen (CN)

(72) Inventors: Yuxin Tang, Shenzhen (CN); Xianjie Liu, Shenzhen (CN); Runbin Yan, Shenzhen (CN); Grace Zhou, Shenzhen (CN)

(21) Appl. No.: 17/635,537

(22) PCT Filed: Aug. 16, 2019
(86) PCT No.: PCT/CN2019/100956

§ 371 Date: Feb. 15, 2022

(51) Int. Cl. A61K 39/245; A61K 39/00; A61P 35/00; A61K 48/00

(52) U.S. Cl. CPC A61K 39/245; A61K 39/001171; A61P 35/00; A61K 48/005; A61K 2039/55527

(57) ABSTRACT

Disclosed is a pharmaceutical composition comprising oncolytic herpes simplex virus expressing IL12 and PD-1 antibody for treatment of cancer through systemic administration.

### TECHNICAL FIELD

The present invention is related to a pharmaceutical composition for treating cancer, comprising an oncolytic herpes simplex virus (oHSV) and formulated for systemic delivery. The present invention is also related to a method for treating cancer comprising administering an oncolytic herpes simplex virus (oHSV) to a subject, in which the oHSV is systemically administered to the subject.

### BACKGROUND

Oncolytic virus therapy is a novel tumor treatment method that utilizes virus-specific replication in tumor cells to kill tumor cells and stimulate the body to produce a specific anti-tumor immune response. Compared with other tumor treatment methods, oncolytic virus therapy has the characteristics of high replication efficiency, well killing effect and small side effects, and has become a hot spot in the field of cancer treatment research.

Oncolytic herpes simplex viruses (oHSV) are being extensively investigated for treatment of solid tumors. As a group, they pose many advantages over traditional cancer therapies. Specifically, oHSV usually embody a mutation that makes them susceptible to inhibition by some aspect of innate immunity. As a consequence, they replicate in cancer cells in which one or more innate immune responses to infection are compromised but not in normal cells in which the innate immune responses are intact. oHSV are usually delivered directly into the tumor mass in which the virus can replicate. Because it is delivered to the target tissue rather than systemically, there are no side effect characteristics of anti-cancer drugs.

However, intratumoral injection of oncolytic virus is mainly used to treat solid tumors or localized tumors. Patients suffering from a tumor not generally accessible for intratumor injection by a physician, e.g., brain cancer or metastatic tumor, can barely benefit from current oncolytic virus therapy. Systemic delivery has the opportunity to infect all tumors and is especially important for metastatic tumors or hematological tumors. However, many hurdles are to be overcome before systemic delivery is clinically available for oHSV. First, systemically administered oncolytic viruses are easily diluted by circulating fluids, including blood, to reduce the concentration of oncolytic viruses reaching target cells, thereby reducing the efficacy of lysing tumor cells; if the dose is increased to increase the concentration of the virus reaching the target site, it may increase the body's inflammatory response. Second, intravenous administration is susceptible to interference by circulating blood components, such as immunomodulation, antibody neutralization, and complement activation, resulting in inactivation of the oncolytic virus or rapid clearance. Moreover, the oncolytic virus also passes through the tissue vascular endothelial cell layer, avoids transcytosis of endothelial cells, and is then transduced into target cells. Finally, intravenous administration may also cause systemic spread, leading to serious non-targeted infections and the like.

Many studies have attempted to deliver oHSV systemically. Du, Wanlu, et al. “Stem cell-released oncolytic herpes simplex virus has therapeutic efficacy in brain metastatic melanomas.” Proceedings of the National Academy of Sciences (2017):201700363 reported the utility of mesenchymal stem cells (MSCs) as oncolytic virus carriers to disseminated brain lesions. They armed MSC with different oHSV variants (MSC-oHSV) and found that intracarotid administration of MSC-oHSV, but not of purified oHSV alone, effectively tracks metastatic tumor lesions and significantly prolongs the survival of brain tumor-bearing mice.

Kanzaki, A, et al. “Antitumor efficacy of oncolytic herpes simplex virus adsorbed onto antigen-specific lymphocytes.” Cancer Gene Therapy 19.4 (2012):292-298 adsorbed oncolytic herpes simplex virus-1 mutant R3616 onto lymphocytes harvested from mice with acquired antitumor immunity. They administered adsorbed R3616 to peritoneally disseminated tumors and analyzed the efficacy of this treatment. Mice administered adsorbed R3616 survived significantly longer than mice administered R3616 adsorbed onto non-specific lymphocytes, or mice administered either virus or tumor antigen-specific lymphocytes alone.

Shikano, T., et al. “High Therapeutic Potential for Systemic Delivery of a Liposome conjugated Herpes Simplex Virus.” Current Cancer Drug Targets 11.1(2011):111-122 encapsulated oncolytic HSV in liposomes. The infectious properties of the herpes simplex virus type 1 (HSV-1) mutant, hrR3, with or without liposomes in the presence of neutralizing antibodies were evaluated using replication and cytotoxicity assays in vitro. To evaluate the efficacy of intravascular virus therapy with liposomes in the presence of neutralizing antibodies, immunized mice bearing multiple liver metastases were intraportally or peritoneally administered hrR3 or hrR3 complexed with liposomes. Results showed that anti-HSV antibodies attenuated the infectiousness and cytotoxicity of hrR3, whereas hrR3/liposome complexes were not attenuated by these anti-HSV antibodies. Although the survival rate of non-immunized mice treated with hrR3 alone was similar to that of mice treated with the hrR3/liposome complexes, the survival rates of immunized mice treated with hrR3 alone were significantly reduced compared to mice treated with the hrR3/liposome complexes. They concluded that systemic intravascular delivery of hrR3/liposome complexes in the presence of pre-existing neutralizing antibodies is effective to treat multiple liver metastases.

Yoo, J. Y., et al. “Copper Chelation Enhances Antitumor Efficacy and Systemic Delivery of Oncolytic HSV.” Clinical Cancer Research 18.18(2012):4931-4941 showed that combination of systemic ATN-224 (a copper chelating agent) and oHSV significantly reduced tumor growth and prolonged animal survival. Immunohistochemistry and DCE-MRI imaging confirmed that ATN-224 reduced oHSV-induced blood vessel density and vascular leakage. Copper at physiologically relevant concentrations inhibited oHSV replication and glioma cell killing and this effect was rescued by ATN-224. ATN-224 increased serum stability of oHSV and enhanced the efficacy of systemic delivery. The study showed that combining ATN-224 with oHSV, significantly increased serum stability of oHSV and greatly enhanced its replication and antitumor efficacy.

Although extended studying and testing in pre-clinical and clinical setting, an unmet need continues to exist for methods for systemic delivery of oncolytic herpes simplex viruses to treat tumors in the presence of intact immune systems.

### SUMMARY

The present inventors were surprised to find that a single injection of oHSV alone via tail vein of tumor-bearing mice significantly inhibit tumor growth without causing significant toxicity. Distribution analysis showed that virus selectively enriched in the tumors up to 4 weeks or longer after the single injection.

In one aspect, the present disclosure relates to a pharmaceutical composition for treatment of cancer in a subject, comprising a therapeutically effective amount of an oncolytic herpes simplex virus (oHSV), wherein the oHSV is modified compared to wild type herpes simplex virus to have (i) a deletion between the promoter of U_{L}56 gene and the promoter of Us1 gene, and (ii) an addition of a heterologous nucleic acid sequence encoding an immunostimulatory agent and/or an immunotherapeutic agent, and wherein the pharmaceutical composition is formulated for systemic delivery to the subject.

In another aspect, the present disclosure relates to a method treatment of cancer in a subject, comprising systemically administering to the subject a pharmaceutical composition comprising a therapeutically effective amount of an oncolytic herpes simplex virus (oHSV), wherein the oHSV is modified compared to wild type herpes simplex virus to have (i) a deletion between the promoter of U_{L}56 gene and the promoter of Us1 gene, and (ii) an addition of a heterologous nucleic acid sequence encoding an immunostimulatory agent and/or an immunotherapeutic agent.

In another aspect, the present disclosure relates to an oncolytic herpes simplex virus (oHSV) for use in a method for treatment of cancer in a subject, wherein the oHSV is modified compared to wild type herpes simplex virus to have (i) a deletion between the promoter of U_{L}56 gene and the promoter of Us1 gene, and (ii) an addition of a heterologous nucleic acid sequence encoding an immunostimulatory agent and/or an immunotherapeutic agent, and wherein the oHSV is systemically administered to the subject.

In some embodiments, the oHSV or the pharmaceutical composition is systemically delivered to the subject not more than twice. In some embodiments, the oHSV or the pharmaceutical composition is systemically delivered to the subject only once.

In some embodiments, the oHSV or the pharmaceutical composition is systemically delivered to the subject without causing significant toxicity.

In some embodiments, the oHSV is freely distributed in the composition. In some embodiments, the oHSV is not encapsulated or supported by a carrier.

In some embodiments, the oHSV is not administered in combination with a second active agent that prevents from the subject's immune response against the oHSV. In some embodiments, the second active agent is a copper chelating agent.

Other aspects of the invention will be readily available from reading the description below.

### BRIEF DESCRIPTION OF DRAWINGS

FIG. 1. Panel A. Biodistribution. Mice in groups of 4 with A549 tumors averaging 70 mm³ were single-injected intratumorally on days 1 with 1×10⁷ pfu of T3011. The volume of virus or PBS injected into tumors was 100 μl. Viral DNA extracted from indicated organs was quantified by qRT-PCR. Panel B. Efficacy. Mice in groups of 6 with A549 tumors averaging 70 mm³ were single-injected intratumorally with 1×10⁵ or 1×10⁷ pfu of T3011. The volume of virus or PBS injected into tumors was 100 μl. Tumor volumes were measured as indicated day after injection. Error bars represent ±SEM for each group.

FIG. 2. Panel A. Biodistribution. Mice in groups of 4 with KYSE30 tumors averaging 108 mm³ were single-injected via tail intravenous on day 1 with 1×10⁷ pfu of T3011. The volume of virus or PBS injected into tumors was 100 μl. Viral DNA extracted from indicated organs was quantified by qRT-PCR. Panel B. Efficacy. Mice in groups of 7 with KYSE30 tumors averaging 108 mm³ were single-injected via tail intravenous on day 1 with 1×10⁵ or 1×10⁷ pfu of T3011. The volume of virus or PBS injected into tumors was 100 Tumor volumes were measured at indicated days after injection. Error bars represent ±SEM for each group.

FIG. 3. Panel A. Biodistribution. Mice in groups of 4 were implanted with HCT116 on left flank, ECA109 on right flank. The mice were received 1×10⁷ of T3011 PFU via tail intravenous injection once the tumor volumes averaged 160 mm³ (HCT116) and 140 mm³ (ECA109). Panel B. Efficacy. Mice in groups of 6 were implanted with HCT116 on left flank, ECA109 on right flank. The mice were received 1×10⁵ or 1×10⁷ PFU of T3011 via tail intravenous injection once the tumor volumes averaged 160 mm³ (HCT116) and 140 mm³ (ECA109). The volume of virus or PBS injected into tumors was 100 Tumor volumes were measured as indicated day after injection. Error bars represent ±SEM for each group.

### DETAILED DESCRIPTION OF THE INVENTION

### Definitions

It is to be noted that the term “a” or “an” entity refers to one or more of that entity; for example, “an exosome,” is understood to represent one or more exosomes. As such, the terms “a” (or “an”), “one or more,” and “at least one” can be used interchangeably herein.

As used herein, the term “systemic administration” or “systemically administered” refers to the occurrence/production of a systemic effect by a certain route of administration, for example, by absorption into the blood for systemic action. Among them, the administration route includes intravenous administration (such as intravenous injection), intramuscular administration (such as intramuscular, subcutaneous, intradermal injection), digestive tract administration (such as oral administration), mucosal administration (such as sublingual administration, oral spray, oral film, eye drops, rectal and vaginal suppositories).

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art.

As used herein, the terms “treat” or “treatment” refer to both therapeutic treatment and prophylactic or preventative measures, wherein the object is to prevent or slow down (lessen) an undesired physiological change or disorder, such as the progression of tumor. Beneficial or desired clinical results include, but are not limited to, alleviation of symptoms, diminishment of extent of disease, stabilized (i.e., not worsening) state of tumor, inhibition of tumor growth, reducing the volume of the tumor, delay or slowing of tumor progression, amelioration or palliation of the tumor state, and remission (whether partial or total), whether detectable or undetectable. Those in need of treatment include those already have a tumor as well as those who are prone to have a tumor.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. The subject herein is preferably a human.

As used herein, phrases such as “to a patient in need of treatment” or “a subject in need of treatment” includes subjects, such as mammalian subjects, that would benefit from administration of a composition of the present disclosure used, e.g., for detection, for a diagnostic procedure and/or for treatment.

By “therapeutically effective amount” it is meant that the oncolytic virus and/or the exosome of the present disclosure is administered in an amount that is sufficient for “treatment” as described above. The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

As used herein, the term “tumor” refers to a malignant tissue comprising transformed cells that grow uncontrollably (i.e., is a hyperproliferative disease). Tumors include leukemias, lymphomas, myelomas, plasmacytomas, and the like; and solid tumors. Examples of solid tumors that can be treated according to the invention include but are not limited to sarcomas and carcinomas such as melanoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, gastric carcinoma and forestomach carcinoma.

As used herein, an “antibody” or “antigen-binding polypeptide” refers to a polypeptide or a polypeptide complex that specifically recognizes and binds to one or more antigens. An antibody can be a whole antibody and any antigen binding fragment or a single chain thereof. Thus, the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. The term antibody also encompasses polypeptides or polypeptide complexes that, upon activation possess antigen-binding capabilities.

The terms “antibody fragment” or “antigen-binding fragment”, as used herein, is a portion of an antibody such as F(ab′)2, F(ab)z, Fab′, Fab, Fv, scFv and the like. Regardless of structure, an antibody fragment binds with the same antigen that is recognized by the intact antibody. The term “antibody fragment” includes aptamers, isomers, and diabodies. The term “antibody fragment” also includes any synthetic or genetically engineered protein that acts like an antibody by binding to a specific antigen to form a complex.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-ld) antibodies (including, e.g., anti-ld antibodies to LIGHT antibodies disclosed herein). Immunoglobulin or antibody molecules of the disclosure can be of any type (e.g. IgG, IgE, IgM, IgD, IgA, and IgY), class (e.g., lgGi, lgG2, IgG3, lgG4, IgA1 and IgA2) or subclass of immunoglobulin molecule.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

The term “IL-12” as used herein refers to “interleukin 12” which is a cytokine with potent antitumor effects. Thus IL-12 induces a TH-1 type immune response, which may provide a durable antitumor effect. IL-12 has been reported to have in vivo anti-angiogenic activity, which may also contribute to its antitumor effects. Lastly IL-12 has been reported to stimulate the production of high levels of IFN-γ, which has multiple immunoregulatory effects including the capacity to stimulate the activation of CTLs, natural killer cells, and macrophages and to induce/enhance the expression of class II MHC antigens. IFN-γ plays a significant role in the process of inducing T-cell migration to tumor sites. Increases in the intratumoral levels of IFN-γ correlated with a decrease in the size of the tumor burden.

Programmed Cell Death 1 (PD-1) is a 50-55 kDa type I transmembrane receptor originally identified by subtractive hybridization of a mouse T cell line undergoing apoptosis. A member of the CD28 gene family, PD-1 is expressed on activated T, B, and myeloid lineage cells. Human and murine PD-1 share about 60% amino acid identity with conservation of four potential N-glycosylation sites and residues that define the lg-V domain. PD-1 negatively modulates T cell activation, and this inhibitory function is linked to an immunoreceptor tyrosine-based inhibitory motif (ITIM) of its cytoplasmic domain. Disruption of this inhibitory function of PD-1 can lead to autoimmunity.

It will also be understood by one of ordinary skill in the art that modified genomes as disclosed herein may be modified such that they vary in nucleotide sequence from the modified polynucleotides from which they were derived. For example, a polynucleotide or a nucleotide sequence derived from a designated DNA sequence may be similar, e.g. have a certain percent identity to the starting sequence, e.g., it may be 60%, 70%, 75%, 80%, 85%, 90%, 95%, 98%, or 99% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions insertions, or deletions relative to the starting sequence.

Oncolytic Herpes Simplex Virus

In the present disclosure, the oHSV is modified compared to wild type herpes simplex virus to have (i) a deletion between the promoter of U_{L}56 gene and the promoter of Us1 gene, and (ii) an addition of a heterologous nucleic acid sequence encoding an immunostimulatory agent and/or an immunotherapeutic agent. A detailed description of the oHSV suitable for systemic delivery according to the present invention is given in WO 2017/181420 (see para. [0040] to [0092], the entire disclosure of which is incorporated herein by reference). The present disclosure expects that all the recombinant oHSVs as mentioned in the above stated WO document can be used with the present disclosure.

In some embodiments, the oHSV is a genetically engineered HSV-1 F strain which has a deletion between the promoter of U_{L}56 gene and the promoter of Us1 gene and expresses both IL-12 and an anti-PD-1 antibody (also referred to as T3011 in the present disclosure).

Pharmaceutical Compositions

In some embodiments of the present disclosure, the oHSV as identified above is formulated to a pharmaceutical composition for systemic delivery to a subject.

In the present disclosure, the oHSV in the composition is freely distributed in the pharmaceutical composition. By “freely distributed” it is meant that the virus is evenly dispersed in the composition, e.g., a sterile injectable solution.

In some embodiments, the oHSV in the composition is not in any way conjugated with a carrier, for example, a cell. In some embodiments, the oHSV is not conjugated with a mesenchymal stem cell. In some embodiments, the oHSV is not absorbed on an antigen-specific lymphocyte.

In some embodiments, the oHSV in the composition is not in any way encapsulated in a carrier, for example, a liposome.

In some embodiments, the oHSV in the composition is not delivered in combination with a second active agent that prevents from the subject's immune response against the oHSV.

Sterile aqueous media that can be employed will be known to those of skill in the art in light of the present disclosure. For example, one dosage may be dissolved in 1 mL of isotonic NaCl solution and either added to 1,000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA.

Sterile injectable solutions are prepared by incorporating the oHSV in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the oHSV plus any additional desired ingredient from a previously sterile-filtered solution thereof.

Methods and Therapies

Another aspect of the disclosure provides a method for treatment of cancer in a subject comprising systemically administering to the subject in need thereof a therapeutically effective amount of the oHSV or the composition of the present invention.

In some embodiments, the recombinant oHSV is systemically administered only once.

In some embodiments, the recombinant oHSV is systemically administered not more than twice. In some embodiments, the oHSV is systemically administered more than twice, for example, 3 times, 4 times or more. In such instances, it is contemplated that one may administer the subject with each administration within about 12 to 72 hrs of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, the oHSV or pharmaceutical composition is administered systemically which is selected from intravenously, intramuscularly, orally, percutaneously and intracutaneously. In some embodiments, the oHSV or the pharmaceutical composition is preferably administered intravenously.

The present disclosure is contemplated to treat various tumors, whether it is a solid tumor or a non-solid tumor. Examples of solid tumors that can be treated according to the invention include but are not limited to leukemias, lymphomas, myelomas, plasmacytomas, sarcomas and carcinomas such as melanoma, fibrosarcoma, myosarcoma, liposarcoma, chondrosarcoma, osteogenic sarcoma, chordoma, angiosarcoma, endothelio sarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, gastric carcinoma and forestomach carcinoma.

In some embodiments, the method of the disclosure contemplated treatment of a tumor that is not accessible by intratumor injection by a physician, for example, a brain tumor or a metastatic tumor.

In some embodiments, the method of the disclosure is preferably used for treating a subject with a metastatic tumor or metastatic tumors.

In some embodiment, the method of the disclosure exhibits no significant or life-threatening toxicity. As demonstrated in the animal experiments shown below, no mice died throughout the experiments.

### EXAMPLES

We describe the development of oHSV therapy that can be administered systemically.

We also identified a murine tumor relatively resistant to the oncolytic activity of murine T3 series of oHSV which is a systemically administered recombinant oncolytic HSV-1 F strain comprising a modified HSV-1 genome and simultaneously expresses IL-12 and anti-PD-1 antibodies (also referred to as “T3011” hereinafter). The modification comprises a deletion between the promoter of U_{L}56 and the promoter of Us1 of a wild-type HSV-1 genome such that (i) one copy of all double-copy genes is absent and (ii) sequences required for expression of all existing open reading frames (ORFs) in the viral DNA after the deletion are intact.

The examples show that the composition comprising T3011 by intravenous injection is not completely cleared by neutralizing antibodies or immune cells in the innate immune system, and the T3011 is able to reach tumor cells and exert an anti-tumor effect that is not significantly different from intratumoral injection.

Materials and Methods

Tumors. Tumors used in this study were human pulmonary carcinoma (A549), Human Esophageal Squamous Cell Carcinoma (KYSE30), Human Colorectal Carcinoma (HCT116) and Human Esophageal Cancer (ECA109).

oHSV construction. The construct of an exemplary oHSV (such as T3011) involves a systemically administered recombinant oncolytic Herpes Simplex Virus type 1 comprising (a) a modified HSV-1 genome wherein the modification comprises a deletion between the promoter of U,56 gene and the promoter of Us1 gene of a wild-type HSV-1 genome such that (i) one copy of all double-copy genes is absent and (ii) sequences required for expression of all existing open reading frames (ORFs) in the viral DNA after the deletion are intact: and (b) a heterologous nucleic acid sequence encoding an immunostimulatory and/or immunotherapeutic agent, wherein the heterologous nucleic acid sequence is stably incorporated into at least the deleted region of the modified HSV-1 genome. Where only one heterologous nucleic acid sequence encoding an immunostimulatory or immunotherapeutic agent is inserted, the heterologous nucleic acid sequence is preferably incorporated into the deleted region of the genome. Where more than one heterologous nucleic acid sequences encoding immunostimulatory and/or immunotherapeutic agents are incorporated, a first heterologous nucleic acid sequences is preferably inserted into the deleted region of the genome. A second or further heterologous nucleic acid sequences may be inserted into the L component of the genome. A more detailed description of the construction and properties of the oncolytic herpes simplex virus (oHSV) is available from WO2017/181420.

Results

Single-Injected Intratumorally of T3011 Inhibited A549 Tumor Growth

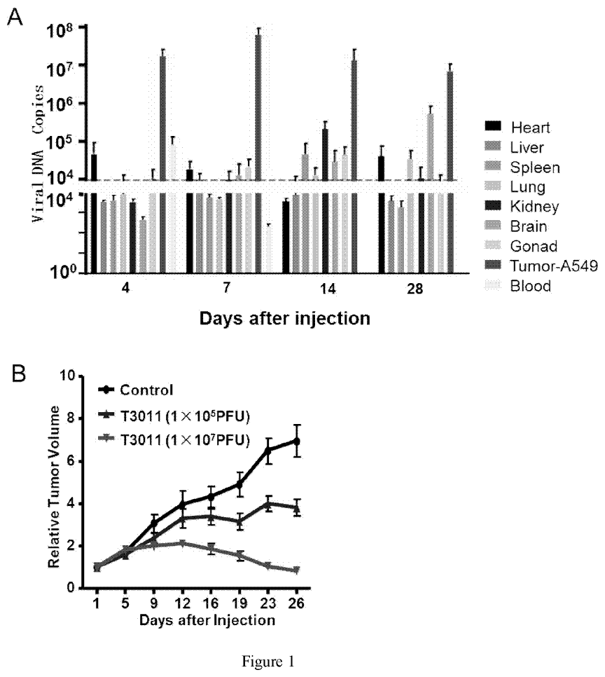

In the first step of this series of experiments, oHSV T3011 was constructed as described in Materials and Methods. Then mice in groups of 4 with A549 tumors averaging 70 mm³ were single-injected intratumorally on days 1 with 1×10⁷ pfu of T3011. The volume of virus or PBS injected into tumors was 100 μl. 4 days, 7 days, 14 days, 28 days after the injection, viral DNA extracted from indicated organs was quantified by qRT-PCR (FIG. 1A).

The results of this section showed, after a single intratumoral injection of T3011, viral DNA molecules were abundantly enriched in A549 tumors and begin to express its genes. In addition, some viruses are also enriched in other organs such as heart, liver, spleen, lung, kidney, brain, gonads and blood. Due to the lack of ability to express genes in normal tissues, viral DNA molecules will stay as long as the host cells survive.

Next, mice in groups of 6 with A549 tumors averaging 70 mm³ were intratumoral single injected with 1×10⁵ or 1×10⁷ PFU of T3011. The volume of virus or PBS (Control) injected into tumors was 100 μl. Then tumor volumes were measured every 3 or 4 days until 26 days after injection (FIG. 1B).

The results of this section showed that the A549 tumor volume in Control and in the mice injected with 1×10⁵ PFU of T3011 increased gradually after injection. The tumor volume of the mice injected with 1×10⁵ PFU of T3011 increased more slowly than that of the Control. The tumor volume of the mice injected with 1×10⁷ PFU of T3011 first increases gradually and then gradually decreases. After 26 days of injection, the tumor volume of the Control was the largest, followed by the tumor volume of the mice injected with 1×10⁵ PFU of T3011, and the tumor volume of the mice injected with 1×10⁷ PFU of T3011 was the smallest.

This series of experiments showed that the viral DNA molecules were most distributed in tumors after intratumoral injection of T3011. Some viral DNA molecules were also distributed in tissues such as heart, liver, spleen, lung, kidney, brain, gonads and blood. At the same time, intratumoral injection of T3011 can effectively inhibit tumor growth, and the anti-tumor effect of 1×10⁷ PFU of T3011 is better than 1×10⁵ PFU of T3011.

Single-Injected Via Tail Intravenous of T3011 Inhibited KYSE30 Tumor Growth

The objective of the series of experiments was to test whether intravenous of T3011 can achieve the same effect as intratumoral injection.

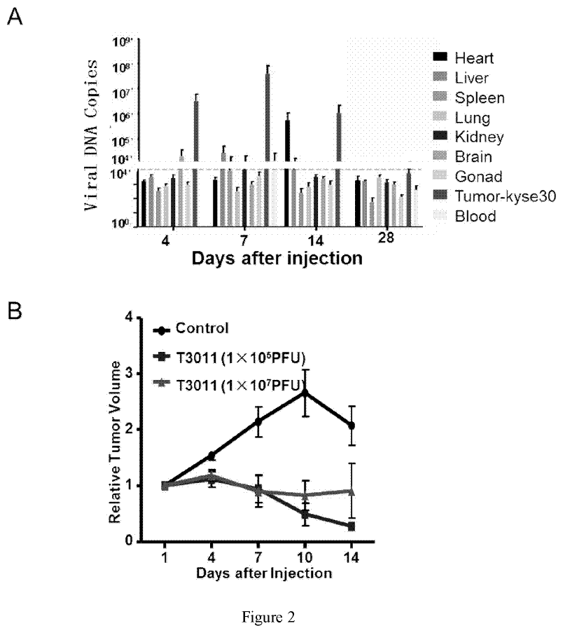

To this end, first of all, mice in groups of 4 with KYSE30 tumors averaging 108 mm³ were single-injected via tail intravenous on day 1 with 1×10⁷ PFU of T3011. The volume of virus or PBS injected into tumors was 100 μl. 4 days, 7 days, 14 days, 28 days after the injection, viral DNA extracted from indicated organs was quantified by qRT-PCR. As illustrated in FIG. 2A, after a single intravenous injection of T3011, viral DNA molecules were abundantly enriched in KYSE30 tumors and begin to express its genes. In addition, some viruses are also enriched in other organs such as heart, liver, spleen, lung, kidney, brain, gonads and blood. Due to the lack of ability to express genes in normal tissues, viral DNA molecules will stay as long as the host cells survive.

Then, mice in groups of 7 with KYSE30 tumors averaging 108 mm³ were single-injected via tail intravenous on day 1 with 1×10⁵ or 1×10⁷ PFU of T3011. The volume of virus or PBS (Control) injected into tumors was 100 μl. Then tumor volumes were measured every 3 or 4 days until 14 days after injection. The results showed that the tumor volume in control increased gradually after injection, reached the maximum on the 10th day after the injection, and then began to decrease. The tumor volume of mice injected with 1×10⁵ PFU of T3011 and 1×10⁷ PFU of T3011 did not change significantly after injection. On the 7th day after injection, the tumor volume of mice injected with 1×10⁵ PFU of T3011 began to decrease gradually, and the tumor volume of mice injected with 1×10⁷ PFU of T3011 began to increase slowly. After 26 days of injection, the tumor volume of the Control was the largest, followed by the tumor volume of mice injected with 1×10⁷ PFU of T3011, and the tumor volume of mice injected with 1×10⁵ PFU of T3011 was the smallest (FIG. 2B).

This series of experiments showed that, similar to the results of intratumoral injection of T3011, after a single intravenous injection of T3011, viral DNA molecules were most distributed in tumors, and some viral DNA molecules were also distributed in tissues such as heart, liver, spleen, lung, kidney, brain, gonads and blood. At the same time, a single intravenous injection of 1×10⁵ PFU of T3011 can effectively inhibit tumor growth.

Single-Injected Via Tail Intravenous of T3011 Inhibited HCT116 and ECA109 Tumors Growth

Some experiments were carried out to verify whether intravenous injection of T3011 effectively inhibited tumor growth when there were two tumors in the body.

Firstly, mice in groups of 4 were implanted with HCT116 on left flank, ECA109 on right flank. The mice were received 1×10⁷ of T3011 PFU via tail intravenous injection once the tumor volumes averaged 160 mm³ (HCT116) and 140 mm³ (ECA109). The volume of virus or PBS injected into tumors was 100 μl. 2 days, 4 days, 7 days, 14 days, 28 days, 42 days and 56 days after the injection, viral DNA extracted from indicated organs was quantified by qRT-PCR.

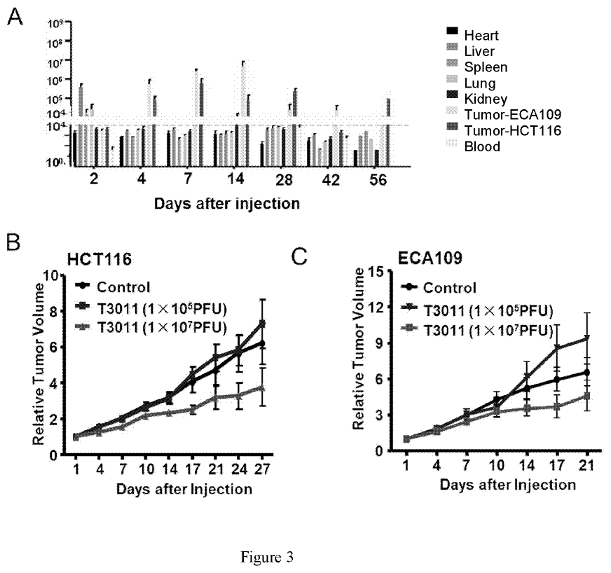

The results (FIG. 3A) of this section showed, after a single intravenous injection of T3011, viral DNA molecules were most distributed in HCT116 tumor and ECA109 tumor and began to express its genes. In addition, some viruses were also distributed in other organs such as heart, liver, spleen, lung, kidney and blood. Due to the lack of ability to express genes in normal tissues, viral DNA molecules will stay as long as the host cells survive.

Next, mice in groups of 6 were implanted with HCT116 on left flank, ECA109 on right flank. The mice were received 1×10⁵ or 1×10⁷ PFU of T3011 via tail intravenous injection once the tumor volumes averaged 160 mm³ (HCT116) and 140 mm³ (ECA109). The volume of virus or PBS injected into tumors was 100 μl. Then tumor volumes were measured every 3 or 4 days until 21 or 27 days after injection.

The results of this section showed that the tumor volume in every group increased gradually after injection. FIG. 3B shows that the HCT116 tumor volume of mice injected with 1×10⁵ PFU of T3011 increased almost as fast as the HCT116 tumor volume of the control, while the HCT116 tumor volume of mice injected with 1×10⁷ PFU of T3011 increased more slowly than that of the Control. After 27 days of injection, the HCT116 tumor volume of the mice injected with 1×10⁵ PFU of T3011 was not significantly different from that of the Control, but the HCT116 tumor volume of mice injected with 1×10⁷ PFU of T3011 was significantly smaller than that of mice injected with 1×10⁵ PFU of T3011 and Control. In FIG. 3C, within 10 days of injection, the ECA109 tumor volume of mice injected with 1×10⁵ PFU of T3011 or 1×10⁷ PFU of T3011 increased almost as fast as the ECA109 tumor volume of the control. After 10 days of injection, mice injected with 10⁵ PFU of T3011 had the fastest increase in tumor volume of ECA109, followed by the tumor volume of the Control, and the slowest increase in ECA109 tumor volume of mice injected with 10⁵ PFU of T3011. After 21 days of injection, the ECA109 tumor volume of the mice injected with 1×10⁵ PFU of T3011 was the largest, followed by the tumor volume of the Control, and the ECA109 tumor volume of mice injected with 1×10⁷ PFU of T3011 was the smallest.

The results of this series of experiments showed, when there were two tumors in the body, the viral DNA molecules were most distributed in the tumors after intravenous injection of T3011. Some viral DNA molecules were also distributed in other organs such as heart, liver, spleen, lung, kidney and blood. In addition, a single intravenous injection of 1×10⁵ PFU of T3011 was not effective in inhibiting the growth of any one tumor when more than one tumor was contained in the body. However, increasing the single intravenous dose of T3011 to 1×10⁷ PFU can effectively inhibit the growth of each tumor in both tumors.

In the course of each biodistribution experiment, no experimental mice died, indicating that intravenous T3011 is safe and causing no significant toxicity.

The results of the above experiments show that, similar to the results of intratumoral injection of T3011, viral DNA molecules are also most distributed in tumors and less in other normal tissues when injected intravenously with T3011. Moreover, a single intravenous injection of 1×105 PFU of T3011 can effectively inhibit tumor growth. When the body contains more than one tumor, the effect of simultaneously treating multiple tumors can be achieved by increasing the dose of intravenous T3011.

It should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

1. A pharmaceutical composition for treatment of cancer in a subject, comprising a therapeutically effective amount of an oncolytic herpes simplex virus (oHSV), wherein the oHSV is modified compared to wild type herpes simplex virus to have (i) a deletion between the promoter of U_{L}56 gene and the promoter of Us1 gene, and (ii) an addition of a heterologous nucleic acid sequence encoding an immunostimulatory agent and/or an immunotherapeutic agent, and wherein the pharmaceutical composition is formulated for systemic delivery to the subject.

2. The pharmaceutical composition of claim 1, wherein the immunostimulatory agent is selected from a group consisting of GM-CSF, IL 2, IL 5, IL 12, IL 15, IL 24 and IL 27.

3. The pharmaceutical composition of claim 1, wherein the immunotherapeutic agent is selected from a group consisting of an anti-PD-1 agent and an anti-CTLA-4 agent.

4. The pharmaceutical composition of claim 1, wherein the oHSV expresses both an immunostimulatory agent and an immunotherapeutic agent.

5. The pharmaceutical composition of claim 1, wherein the oHSV expresses IL-12 and an anti-PD-1 antibody.

6. The pharmaceutical composition of claim 1, wherein the oHSV is originated from herpes simplex virus serotype 1 (HSV-1).

7. The pharmaceutical composition of claim 6, wherein the HSV-1 is selected from strains F, KOS, and 17.

8. The pharmaceutical composition of claim 7, wherein the HSV-1 is F strain of HSV-1.

9. The pharmaceutical composition of claim 1, wherein the oHSV contains a deletion of nucleotide positions 117005 to 132096 in the genome of F strain of HSV-1.

10. The pharmaceutical composition of claim 1, wherein the oHSV is not encapsulated within or conjugated by a carrier.

11. The pharmaceutical composition of claim 1, wherein the pharmaceutical composition is administered to the subject intravenously.

12. The pharmaceutical composition of claim 1, wherein the cancer is a solid cancer.

13. The pharmaceutical composition of claim 12, wherein the cancer is selected from a group consisting of leukemias, lymphomas, myelomas, plasmacytomas, melanoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, gastric carcinoma and forestomach carcinoma.

14. The pharmaceutical composition of claim 12, wherein the cancer is not accessible through intratumor injection by a physician.

15. The pharmaceutical composition of claim 1, wherein the subject is human.

16. The pharmaceutical composition of claim 1, wherein the pharmaceutical composition is systemically delivered to the subject not more than twice.

17. The pharmaceutical composition of claim 1, wherein the pharmaceutical composition is systemically delivered to the subject only once.

18. The pharmaceutical composition of claim 1, wherein the pharmaceutical composition is systemically delivered to the subject without causing significant toxicity.

19. The pharmaceutical composition of claim 1, wherein the oHSV is freely distributed in the composition.

20. The pharmaceutical composition of claim 1, wherein the oHSV is not delivered in combination with a second active agent that prevents from the subject's immune response against the oHSV.
